(19) United States
(12) Patent Application Publication
(10) Pub. No.: US 2006/0116696 A1
(43) Pub. Date: Jun. 1, 2006

(54) Planar implant and surgical use thereof

(76) Inventors: Eric K. Odermatt, Schaffhausen (CH); Wolfgang Abele, Tuttlingen (DE); Juergen Wegmann, Wurmlingen (DE)

Correspondence Address: NATH & ASSOCIATES, 112 South West Street, Alexandria, VA, 22314, US

(21) Appl. No.: 10/553,443

(22) PCT Filed: Apr. 13, 2004
(86) PCT No.: PCT/EP04/03849

§ 371 Date: Nov. 29, 2005

(30) Foreign Application Priority Data
Apr. 17, 2003 (DE) 103 18 801.0

(51) Int. Cl. A61F 2/02

(52) U.S. Cl. 606/151; 623/23.72; 606/154

(57) ABSTRACT

The invention relates to a planar implant comprising a planar support with two faces, at least one face of the support being provided with an absorbable adhesive layer which is able to adhere to human or animal tissue.

The invention relates to a planar implant and to the surgical use thereof.

Hernias are a frequently encountered condition. They generally involve organs or organ parts protruding from the natural body cavity through a pre-formed or acquired gap. Among external hernias, where the hernial sac is always enclosed by the peritoneum, the most frequently encountered forms are inguinal, umbilical and incisional hernias. The main reasons for hernias occurring are muscle or connective tissue surfaces in connection with overstraining, age-related atonia, congenital weakness of the abdominal wall or inadequate scar formation following a surgical incision (incisional hernia).

In most cases, effective treatment is possible by a surgical procedure in which the hernia content is moved back from the hernial sac into the abdomen and the hernial orifice is closed after implantation of what is called a hernia mesh. Numerous hernia meshes are known. These are generally knitted meshes. They can be made of polypropylene, PTFE, polyurethane or also polyester.

However, there are also other planar implants which are intended to remain at least temporarily in the body, these including, for example, urinary incontinence bands and so-called patches used to cover wound sites or injuries in organs.

To fix them in place, the planar implants are generally sewn onto suitable body parts, or their surface is so strongly structured that mechanical anchoring takes place. This is the case, for example, in urinary incontinence bands, some of which have jagged edge zones with which they anchor in connective tissue and muscle tissue.

The implants can be completely or partially absorbable, by filament material made of absorbable plastics being incorporated, or by the meshes being constructed in several layers, by a mesh made of a non-absorbable material being combined with a mesh made of absorbable material, or by the meshes being produced entirely from absorbable material. Absorbable materials are, in particular, polymers and copolymers of lactide, glycolide, trimethyl carbonate, epsilon caprolactone, polyhydroxybutanoic acid.

The object of the invention is to create planar implants which permit easier and more gentle securing in the body.

This object is achieved by a planar implant comprising a planar support with two faces, at least one face of the support being provided with an absorbable adhesive layer which is able to adhere to human or animal tissue.

An implant provided with such an adhesive layer is self-adhering, so that additional fixing is unnecessary. The absorbable adhesive layer affords a pre-fixing of the implant until the implant has grown onto or into the tissue as intended. The absorption time of the material of the adhesive layer can be suitably adjusted. The adhesive layer ensures a more homogeneous adaptation to the tissue. This results in a more uniform and more complete infiltration with connective tissue.

Various adhesives are known in surgery. Examples that may be mentioned here are adhesives formed from thrombin and fibrinogen, with thrombin intervening actively in the blood coagulation cascade. In view of the problems of BSE and HIV, such adhesives have fallen out of favor somewhat. Adhesives based on cyanoacrylate are also often used, but these are difficult to handle and are also problematic in respect of absorption capacity.

In a particularly preferred embodiment of the invention, the adhesive layer is formed essentially from at least one polymer which carries free aldehyde groups and whose aldehyde groups are able to react with nucleophilic groups of the tissue. Such an adhesive layer forms a covalent adhesive connection with the nucleophilic groups of the tissue, in particular with amino groups, SH— groups and OH— groups. A covalent adhesive connection is in this case formed, for example, by imine bonds, between the aldehyde groups of the polymer and amino groups of the blood, of the serum and especially of the surrounding body tissue. These imine bonds (Schiff bases) are reversible covalent bonds which are stronger than pure ionic bonds and permit good and uniform adherence of the implant to the body tissue. In the case of SH— or OH— groups, the implant according to the invention forms, between the adhesive layer and body parts, adhesive connections in the form of acetal or thioacetal bonds which behave correspondingly to the imine bonds.

One to five percent of all implanted meshes show infections caused by persistent bacteria, which may lead to secondary abscesses. With the use of polymers carrying aldehyde groups, in particular dextran aldehyde, a disinfectant and bactericidal action is exerted on the wound site, and also on the mesh, which action leads to a reduced infection rate and consequently to a reduced short-term to long-term recurrence rate. The anti-infective action can be strengthened by admixture of nano silver particles.

In one embodiment, the adhesive layer consists of only one polymer. In other embodiments, the adhesive layer consists of a combination of different polymers. In further embodiments, the polymer of the adhesive layer has cross-linkages, via which the stability and hardness of the adhesive layer can be adjusted. The degradation time of the adhesive layer can also be increased by addition of cross-linking agents. The polymer in the adhesive layer is normally present in uncrosslinked form. It is also possible to incorporate additives such as softeners. Also, pharmacologically active substances which are released from the adhesive layer to the surrounding tissue and to body fluids, for example growth-promoting substances, wound-healing substances, disinfectants, antibiotics and the like.

The adhesive layer can also only partially cover at least one face of the implant. This may be desirable if only individual fixation points are needed. In general, the face of the implant is completely covered by the adhesive layer. An open structure, in particular a porous structure of the adhesive layer is preferred. The adhesive layer can be applied in such a way that it is also present on the support only at the edges. Depending on the material and the material structure, the incorporation of a possibly critical edge zone of the support can in this way be facilitated and strengthened.

In general, only one face of the support is provided with the adhesive layer. In special cases, the adhesive layer may be provided on both faces of the implant. This is the case when the implant is used to connect body parts by interposition of the implant. The connection of body parts is preferably carried out with absorbable planar implants which ensure apposition through the additionally applied polymer, preferably dextran aldehyde carrying aldehyde groups and/or polyvinyl alcohol carrying aldehyde groups.

It is also possible to provide the adhesive layer on one face of the implant and to provide an anti-adhesive layer on the other face of the implant. This is advantageous especially in hernia meshes in which a good connection with the inner face of the abdominal wall is desired, but a connection with the organs of the abdominal space is to be avoided. The anti-adhesive layer is preferably a closed layer and in particular has a smooth surface.

Materials for anti-adhesive layers are known. Polyvinyl alcohol (PVA), in particular with a molecular weight of 20,000 to 200,000, is especially suitable. The polyvinyl alcohol can, in particular, be cross-linked in a known manner in order to control its absorption time.

The use of polyvinyl alcohol and its preparation for adhesion prophylaxis is described, for example, in WO 02/09789 A2, the content of which is referred to here. Carboxymethylcellulose (CMC), if appropriate in conjunction with polyvinyl alcohol, is also suitable as anti-adhesive layer. The anti-adhesive layer can also contain active substances, as mentioned above.

The adhesive layer of the implant according to the invention preferably has an open-cell structure and in particular is absorbent. In this way, the adhesion can be accelerated by uptake of body fluid. The adhesive layer is advantageously hydrophilic. In a particularly preferred embodiment, the adhesive layer is able to take up aqueous fluids by swelling, which has a favorable effect especially in combination with the absorbency.

In a particular embodiment, the adhesive layer has a fibrous structure and is preferably present in the form of a nonwoven, in particular a three-dimensional nonwoven with a fibrous structure which has a total surface area many times greater than the outer surface of the nonwoven. It is also conceivable that the adhesive layer is present in the form of an open-cell planar foam. Here too, the inner surface is many times greater compared to the outer surface. The adhesive layer can also be present in the form of a film or membrane. For example, the adhesive layer can be formed by pressing or rolling of a foam or of a lyophilisate. A further possible production technique is direct knife-coating of the adhesion promoter and spraying onto the planar support.

The adhesive layer advantageously contains water-soluble components and is in particular completely soluble in water. In this way, the adhesive layer can be removed through dissolution as the incorporation of the implant proceeds, so that degradation in situ is not necessary.

The time required for dissolution in the body can also be adjusted by chemical and/or physical measures, in particular by the degree of cross-linking, by freeze/thaw cycles, by varying structure and domain formations.

In a preferred embodiment, the implant is flexible. This accordingly applies also to its adhesive layer. The adhesive layer does not have to be completely dry. It can also at least partially contain water as a softener or may even be present in the form of a hydrogel or gel.

According to a preferred embodiment, the polymer carrying aldehyde groups is an oxidized, in particular bioabsorbable polysaccharide. Possible oxidized polysaccharides are starch, agar, cellulose, alginic acid, xanthan and hyaluronic acid. In a particular embodiment, the polysaccharide is dextran polysaccharide. The polymer of the adhesive layer carrying aldehyde groups can also be a synthetic polymer, in particular polyethylene glycol (PEG), which is preferably branched. In this embodiment, the polyethylene glycol has at least three terminal aldehyde groups, which can form covalent bonds with the nucleophilic groups of the body tissue. A further possible synthetic polymer carrying aldehyde groups is polyvinyl alcohol (PVA), in particular branched polyvinyl alcohol which preferably has at least three terminal aldehyde groups.

In further embodiments, other biocompatible polyols or polyethylene oxide (PEO) can also be provided as the polymer backbone of the polymer carrying aldehyde groups.

In the polymer of the adhesive layer, the aldehyde groups within the molecule can be set apart from the polymer backbone by a spacer. This may be advantageous in particular in oxidized polyethylene glycol or polyvinyl alcohol. The polymers carrying aldehyde groups can be strengthened with polymers carrying no aldehyde groups, such as, in particular, polyvinyl alcohol and/or carboxymethylcellulose.

The proportion of glucose units oxidized to the aldehyde in the dextran polyaldehyde contained in the adhesive layer is advantageously at least 20%, preferably 35 to 100%, and in particular 50 to 85%. By means of a high proportion of glucose units oxidized to the aldehyde, a multiplicity of covalent bonds is obtained and, consequently, a strong adhesive connection between implant and body tissue.

The adhesive layer of the implant according to the invention can be connected to the planar support in different ways. For example, the adhesive layer can be produced as a separate layer or membrane which is then connected to the support of the implant. It is preferable to form the adhesive layer directly on the support. The adhesive layer can be connected to the support by exploiting adhesion properties of the adhesive layer or, if appropriate, by using an additional and in particular absorbable adhesive agent. The adhesive layer can be formed on the support by means of the at least still partially liquid or tacky material of the adhesive layer being brought into contact with the support and then dried. Thus, for example, the support can be immersed in a solution of the polymer of the adhesive layer or can be coated with this solution, and the adhesive layer can then be formed by drying in air or lyophilization. It is also possible to apply the material of the adhesive layer in the form of a sprayed-on layer, in particular a spray-bonded nonwoven, to the support. Other known coating possibilities are also possible. The adhesive layer can be prefabricated by simple drying of solutions. Porous adhesive layers are obtained in particular by lyophilization of solutions, and a highly aerated structure is obtainable if the solutions are foamed prior to lyophilization or if quite large hollow spaces are created by addition of crushed ice prior to the freeze-drying of the solution. One-sided adhesive layers can be created by pressing the planar support onto a lyophilized structure. Two-sided adhesive layer structures can be obtained by pressing the planar support between two for example lyophilized structures.

In preferred embodiments, the adhesive layer, because of its sponge-like structure and porosity and its hydrophilic character, can take up at least 30 times its own weight of fluid. Moreover, the adhesive layer is able to take up at least 4 times its own weight of hemoglobin. In this way, in addition to a good adhesive connection, hemostasis is achieved at the same time if so desired.

The at least one polymer carrying aldehyde groups can be cross-linked with a cross-linking agent before production of the adhesive layer. Possible cross-linking agents are bifunctional amines, in particular diamino acids lysine, ornithine, arginine or triethylene glycol diamine, further multifunctional amines, in particular the polyamino acid polylysine, bifunctional or multifunctional molecules containing SH— or NH₂— groups, in particular cyssteine or polycysteine, or bifunctional or multifunctional thiols, and also peptides. Particular preference is given to chitosan.

In a particular embodiment, the adhesive layer, has a surface structured on at least one side. The structured surface improves the adherence of the adhesive layer to the tissue. Various types of structuring are conceivable, such as a square, jagged, braided, woven or spiral-shaped structure. By means of the structuring, the mechanical friction between tissue and adhesive layer is increased and, after application, the implant holds better at the applied position. The structuring can preferably also be formed by the basic structure of the support, for example of the knit. Thus, fibers of a textile structure of the support can be covered with material forming the adhesive layer. In this case, it is preferable that the open or open-cell structure of the textile support be maintained. It is enough if the individual fibers are covered with adhesive layer material.

The structuring can also be produced by means of suitably structured lyophilization dishes or by means of embossing after production of the implant or of the adhesive layer.

As has already been mentioned, the planar support for the adhesive layer is preferably flexible. It preferably has, on at least one face, an open structure suitable for incorporation of cell tissue. Particularly suitable materials for this purpose are textile materials, preference being given to woven materials, braided materials, drawn-loop knits and, in particular, formed-loop knits. These can be produced from monofilament yarns and/or multifilament yarns which are absorbable and/or non-absorbable. If the implant is to remain permanently in the body, the support is made at least partially of non-absorbable material, specifically in such a way that its function is maintained. Thus, for example, the support can be partially absorbable if there is a need for a certain initial stability which over the course of time, however, is then no longer necessary, for example when the body tissue, as a result of the healing process, is able to assume at least in part the function of the support. If a long-term action of the implant is not needed or not desired, the implant as a whole can preferably be absorbable, so that it disappears with time when it has fulfilled its function.

By virtue of the different formation of its surfaces, the implant according to the invention can be used in a number of ways in surgery. It can be used to cover certain organs or to connect tissue parts to one another. If one-face has an anti-adhesive layer, it can also be used to prevent undesired union of body parts.

The implant according to the invention can also be present in different forms. It is generally present in the form of a flexible planar material. However, it can also have a three-dimensional form, in particular tubular with an outer face and an inner face. It can also have the form of a ring. Especially in the case when the implant has a three-dimensional structure, it can also have dimensional stability, in particular elastic dimensional stability.

Implants of this kind with relative dimensional stability can be formed in particular as connecting parts or strengthening parts for tubular hollow organs such as vessels or sections of the intestine. It is also conceivable to provide the surface of solid materials or linear materials, such as surgical suture material or surgical clips, with a corresponding adhesive layer in order to improve their anchoring, incorporation and infection prevention in the body tissue.

The implant according to the invention can be easily sterilized and, in the state ready for use, it is present in a sterile form, in particular in a sterile package that is opened shortly before the implantation procedure.

Further features of the invention will become clear from the following description of preferred embodiments and examples in conjunction with the dependent claims. Here, the individual features of the invention may be realized alone or in combination with one another. The described embodiments serve to explain and to provide a better understanding of the invention and are not to be regarded as limiting the invention.

### EXAMPLES

### Example 1

Production of a planar implant with an adhesive layer on both faces

Dextran aldehyde is dissolved in bidistilled water at 50° C. The solution is poured into a flat dish in a quantity that just covers the bottom. If too much solution is poured in, the excess is poured back out. A hernia mesh is carefully placed onto the solution. The solution is then lyophilized, with an adhesive layer forming from dextran aldehyde on one face of the hernia mesh. The thickness of the adhesive layer corresponds to the filled level of the dextran aldehyde solution before lyophilization. The adhesive layer has the structure of a nonwoven. Adhesive layers of different density and strength can be produced from solutions with different concentration of dextran aldehyde, 1% strength dextran aldehyde solutions and dextran aldehyde solutions of higher concentration producing essentially closed adhesive layers which gain in strength as the concentration increases.

In a hernia mesh of this kind, only one face of the mesh is secured by the adhesion force of the surface. This can be strengthened still further if the face of the hernia mesh directed away from the adhesive layer is sprayed with a viscous solution of polyvinyl alcohol to obtain an anti-adhesive layer, after which it is dried in a stream of air. A hernia mesh is in this way obtained whose face directed toward the abdominal wall forms a rapid and good adhesive connection with the latter, so that it is not necessary to fix the hernia mesh by suturing or clipping. Because of the anti-adhesion layer, the face of the hernia mesh directed toward the inner face of the abdomen prevents undesired fusion of organs of the abdominal space, at least until the wound healing process is completed.

### Example 2

The first step in example 1 is repeated, except that dextran aldehyde is poured into the dish in a quantity which ensures that, when the hernia mesh is placed in the dish, both faces of the hernia mesh are wetted by the solution. Lyophilization results in a mesh which has an adhesive layer from dextran aldehyde on both faces. This implant makes it possible to connect body tissue surfaces to one another. If the mesh is made from absorbable yarn material, the implant disappears after the tissue parts have fused together.

### Example 3

A dimensionally stable but still elastic tube section made from absorbable biocompatible plastic such as PGA, a terpolymer of lactide (65), TMC (19) and caprolactone (16), a copolymer of L-lactide (86) and TMC (14) and/or polyglycolide lactide (90/10), and having the structure of an elastic tubular lattice, is immersed completely in a 5% strength dextran aldehyde solution, after which the solution is dried in a stream of air. A connecting ring of stable diameter is obtained which is suitable for adhesion of intestinal ends following a partial resection.

### Example 4

Immersion Method

Premilene® meshes (knitted meshes made from monofilament polypropylene) were subjected to three immersions in different concentrations of dextran aldehyde (DA) solution. For this purpose, the meshes were immersed for 60 seconds in the respective solution and then dried in air to constant weight. The results are compiled in Table 1.

TABLE 1
Coating of Premilene meshes with different concentrations of DA solutions (immersion
method). [Percent by weight related to the weight of the uncoated mesh]

| DA | Weight | 1^{st} immersion | | 2^{nd} immersion | | 3^{rd} immersion | |
|---|---|---|---|---|---|---|---|
| solution | uncoated | Weight | % by | Weight | % by | Weight | % by |
| [%] | [mg] | [mg] | weight | [mg] | weight | [mg] | weight |
| 2.5 | 125 | 126.9 | 101.5 | 126.9 | 101.5 | 126.4 | 101.1 |
| 5 | 125.9 | 130 | 103.20 | 130 | 103.3 | 129.9 | 103.2 |
| 7.5 | 126.7 | 137.6 | 108.6 | 142 | 112.1 | 151 | 119.2 |
| 10 | 125.2 | 136.9 | 109.3 | 138.5 | 110.6 | 138.5 | 110.6 |

Whereas the solutions with low concentrations provided only a slight weight increase, the coating with the 7.5% strength DA solution was very-high. The coating was not improved any further with the still higher 10% strength DA solution.

### Example 5

Spray Method:

The meshes were sprayed a total of three times with DA solutions of different concentrations and were then dried to constant weight. The spray device used was a Spray Set from the company Confluent Surgical. The distance between spray nozzle and mesh was 20 cm.

TABLE 2
Coating of Premilene meshes with different concentrations of DA solutions (spray
method). [Percent by weight related to the weight of the uncoated mesh].

| DA | Weight | 1^{st} spray | | 2^{nd} spray | | 3^{rd} spray | |
|---|---|---|---|---|---|---|---|
| solution | uncoated | Weight | % by | Weight | % by | Weight | % by |
| [% w/v] | [mg] | [mg] | weight | [mg] | weight | [mg] | weight |
| 2.5 | 118.4 | 122.2 | 103.2 | 123.6 | 104.4 | 123.6 | 104.4 |
| 5 | 123.1 | 125.1 | 101.6 | 126.5 | 102.8 | 127.2 | 103.3 |
| 7.5 | 117.3 | 135.4 | 115.5 | 146.8 | 125.1 | 152.8 | 130.3 |
| 10 | 121.8 | 133.1 | 109.3 | 137.7 | 113.1 | 143.3 | 117.7 |

Compared to the immersion method, the spray method provided a higher degree of coating. In analogy to the immersion method, the highest weight increase was observed with the 7.5% strength DA solution. The mesh-like structure is maintained in all the meshes. The pores are not closed by the dextran aldehyde.

### Example 5a

Different adhesive layers on meshes can be produced as follows:
1. A lyophilized foam with a thickness of 6 mm is produced.
2. A surface measuring 10×15 cm² is cut to size.
3. The corresponding mesh surface of polypropylene is cut to size and sprayed with bidistilled water.
4. The moist mesh is pressed with light pressure onto the lyophilisate and dried on.
5. After drying, the free face of the mesh is again sprayed, and the correspondingly cut PVA prophylaxis film is pressed on and thus secured.

### Examples 6 and 7

Safil® Mesh (Knit Made from Monofilaments and Multifilaments of Polyglycolic Acid):

The Safil meshes were coated with a 10% strength DA solution. Here again, both the immersion method and the spray method were used. The results are compiled in Tables 3 and 4. When three-dimensional mesh structures are sprayed from outside during rotation, a still elastic meshwork with DA is obtained mainly on the outer face.

TABLE 3
Coating of a Safil mesh with a 10% strength DA solution (immersion method).
[Percent by weight related to the weight of the uncoated mesh].

| DA | Weight | 1^{st} immersion | | 2^{nd} immersion | | 3^{rd} immersion | |
|---|---|---|---|---|---|---|---|
| solution | uncoated | Weight | % by | Weight | % by | Weight | % by |
| [%] | [mg] | [mg] | weight | [mg] | weight | [mg] | weight |
| 10 | 92.8 | 107.3 | 115.6 | 107.3 | 115.6 | 111 | 119.6 |

TABLE 4
Coating of a Safil mesh with a 10% strength DA solution (spray method).
[Percent by weight related to the weight of the uncoated mesh].

| DA | Weight | 1^{st} spray | | 2^{nd} spray | | 3^{rd} spray | |
|---|---|---|---|---|---|---|---|
| solution | uncoated | weight | % by | weight | % by | Weight | % by |
| [%] | [mg] | [mg] | weight | [mg] | weight | [mg] | weight |
| 10 | 85.3 | 101.1 | 118.5 | 109.4 | 128.3 | 116.1 | 136.1 |

Again, compared to the immersion method, the spray method provided a greater increase in weight. Compared to the Premilene meshes, the percentage increase in weight is greater in the Safil meshes, i.e. a higher degree of coating of the meshes is possible with Safil.

It is found that the pores of the meshes are not closed by the coating. A closure of the pores can be obtained by increasing the spraying/drying cycles.

In the drawings:

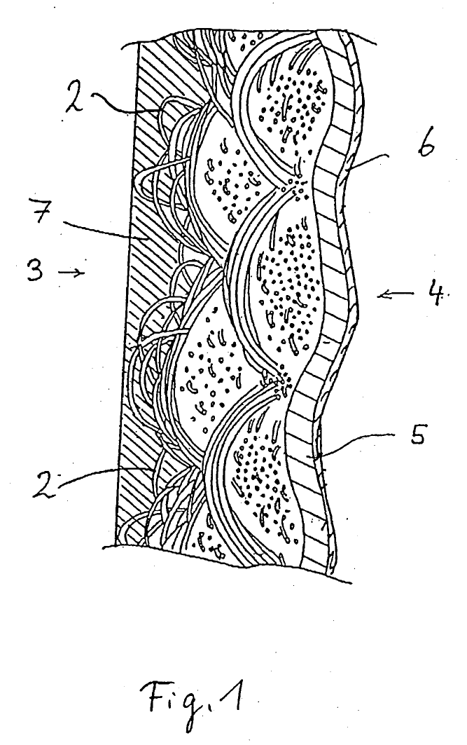

FIG. 1 shows a partial cross section through a hernia mesh with an adhesive layer and an anti-adhesive layer according to example 1,

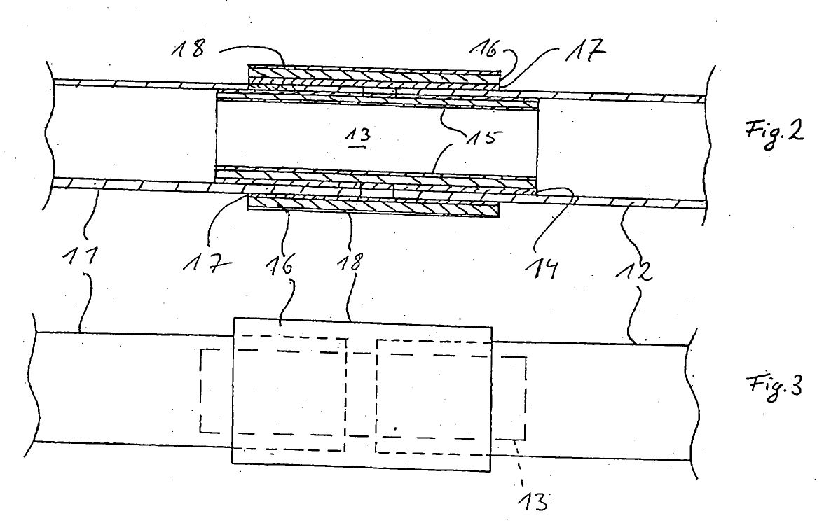

FIG. 2 shows a longitudinal section through a connection between end pieces of the intestine after a resection,

FIG. 3 shows a view of the embodiment according to FIG. 2.

In the embodiment according to FIG. 1, a textile fabric 1 is constructed as a warp knit from multifilament polyethylene terephthalate yarn in the form of a single velour, with velour loops 2 of textured yarn. The knit is porous and flexible and serves as the support of the implant according to the invention. On the velour side 3, the knit as such has an open, three-dimensionally structured surface which, as a result of the velour loops and texturing of the fibers, provides numerous sites distributed substantially uniformly across the surface behind which body cells can engage and grow in. The openings between the yarn loops or the individual fibers are large compared with the size of body cells. This permits the incorporation of a cohesive cell agglomerate.

In accordance with the procedure described in Example 1, an adhesive layer 7 in the form of lyophilized dextran aldehyde is situated on the velour side 3 of the substance. Since the dextran aldehyde solution has penetrated into the surface of the knit prior to the lyophilizacion, a substantially closed but porous adhesive layer is present.

As a result of the weave of the knit, the opposite side 4 of said knit is more dense and rather flat. In addition to the procedure described in Example 1, the knit 1 can on this side have a spray coating 5 of uncrosslinked polyurethane which is connected to those fibers of the knit 1 exposed on the surface and which substantially closes the textile structure on this surface. The spray coating has the structure of a spray-bonded nonwoven. The thickness of this spray-bonded nonwoven is of the order of approximately 1/10th to 1/20th of the total thickness of textile fabric and sealing layer. Situated on the outside of the sealing layer 5 is the anti-adhesion prophylaxis which is mentioned in Example 1 and which is produced by spraying-on of polyvinyl alcohol solution, closes the pores of the spray-bonded nonwoven in a sealing manner and prevents incorporation of cells during the wound-healing phase. If no sealing layer, for example of polyurethane, is needed, the adhesion prophylaxis layer of PVA (6) can be applied directly to the knit.

The embodiment shown can be used as a hernia mesh with good adhesive properties on one face and anti-adhesive properties on the other face, providing a rapid and good connection of the mesh to the abdominal wall and preventing undesired attachment of organs of the abdominal cavity.

Depending on the application requirements, the hernia mesh can consist of monofilament or multifilament yarns. The mesh structure can be thin and light-weight, since the properties needed for the deployment of the mesh can be imparted to it through the coatings. The mesh structure can also be made completely or at least partially of absorbable material.

FIGS. 2 and 3 show schematic representations of the connection between two portions of the intestine after a partial resection. The free ends of the portions 11 and 12 of the intestine are pushed over a tubular implant 13 which is produced basically as described in Example 3. The lattice of the tube section is stiffened by the polymer forming the adhesive layer. In the embodiment described here, only the coating 14 on the outside of the inner tube 13 serves as adhesive layer for connection to the inner face of the intestinal wall. The corresponding coating 15 on the inside has no function and is dissolved by the content of the intestine. A further tube section 16 is pushed over the intestinal connection as an outer tube; it has an adhesive layer 17 only on its inner face, whereas the outer face is covered with an anti-adhesive layer 18 of polyvinyl alcohol. Via the inner tube 13 and the outer tube 16, the ends of the intestine are bonded from the outside and from the inside to the corresponding tubular implants. The anti-adhesive layer 18 ensures that the portion of the intestine located here does not become connected to a further portion of the intestine or to another organ of the abdominal space.

At the same time as the portions of the intestine fuse together, both the adhesive layer and the anti-adhesive layer of the tube sections are dissolved and absorbed. The dextran aldehyde polymer forming the adhesive layer and located inside the lattice is also dissolved over the course of time, so that the flexibility of the tube sections from the lattices accordingly increases. Since the lattices are made of absorbable plastics, e.g. a terpolymer of lactide, TMC and caprolactone (65/19/16) or a copolymer of lactide (86) and TMC (14), the lattice portions acting as textile supports of the implant also dissolve over the course of time, so that, finally, only the fused intestine remains.

1. A planar implant comprising a planar support with two faces, at least one face of the support being provided with an absorbable adhesive layer which is able to adhere to human or animal tissue.

2. The implant as claimed in claim 1, characterized in that the adhesive layer is essentially formed from at least one polymer which carries free aldehyde groups and whose aldehyde groups are able to react with nucleophilic groups of the tissue, and it in particular has anti-infective properties.

3. The implant as claimed in claim 1, characterized in that the adhesive layer at least partially covers, preferably completely covers, the at least one face of the support.

4. The implant as claimed in claim 1, characterized in that the adhesive layer is designed to cover the planar support only around the edges and/or to protrude beyond the edges of the planar support.

5. The implant as claimed in claim 1, characterized in that the adhesive layer is provided on both faces of the support.

6. The implant as claimed in claim 1, characterized in that the support has an adhesive layer on one face and preferably has an anti-adhesive layer on the other face.

7. The implant as claimed in claim 6, characterized in that the anti-adhesive layer has a closed and in particular smooth surface.

8. The implant as claimed in claim 1, characterized in that the adhesive layer is designed as an open layer and is in particular absorbent.

9. The implant as claimed in claim 1, characterized in that the adhesive layer is hydrophilic and in particular is able to take up aqueous fluids by swelling.

10. The implant as claimed in claim 1, characterized in that the adhesive layer is present in the form of a nonwoven, in particular a three-dimensional nonwoven.

11. The implant as claimed in claim 1, characterized in that the adhesive layer is present in the form of an open-cell foam.

12. The implant as claimed in claim 2, characterized in that the polymer carrying aldehyde groups is soluble in water.

13. The implant as claimed in in claim 2, characterized in that the polymer carrying aldehyde groups is an oxidized, in particular bioabsorbable polysaccharide.

14. The implant as claimed in claim 13, characterized in that the oxidized polysaccharide is one from the group comprising starch, cellulose, agar, dextran aldehyde, hyaluronic acid, alginic acid, chondroitin sulfate, and preferably dextran polyaldehyde.

15. The implant as claimed in claim 14, characterized in that the proportion of glucose units oxidized to the aldehyde in the dextran polyaldehyde is at least 20%, preferably 35 to 100%, in particular 50 to 85%.

16. The implant as claimed in claim 2, characterized in that the polymer carrying aldehyde groups is an in particular branched polyethylene glycol with at least three terminal aldehyde groups.

17. The implant as claimed in claim 2, characterized in that the polymer carrying aldehyde groups is an in particular branched polyvinyl alcohol with at least three terminal aldehyde groups.

18. The implant as claimed in claim 2, characterized in that the at least one polymer carrying aldehyde groups is partially cross-linked.

19. The implant as claimed in claim 1, characterized in that the adhesive layer has a structured surface on its outer face.

20. The implant as claimed in claim 1, characterized in that the planar support is porous and flexible, and in particular is formed from a textile material.

21. The implant as claimed in claim 1, characterized in that the support, in particular the textile support, is at least partially absorbable, in particular completely absorbable.

22. The implant as claimed in claim 1, characterized in that one face of the support is provided with at least one anti-adhesive layer which is preferably absorbable.

23. The implant as claimed in claim 21, characterized in that the anti-adhesive layer contains polyvinyl alcohol and/or carboxymethylcellulose, and in particular consists of polyvinyl alcohol.

24. The implant as claimed in claim 1, characterized in that it is designed as a hernia mesh having the adhesive layer on the face which is intended to bear on the abdominal wall, and in that the other face of the hernia mesh preferably has at least one layer which is designed as an anti-adhesive layer and prevents adhesion of body tissue to the mesh.

25. The implant as claimed in claim 1, characterized in that it is designed as a patch which has the adhesive layer on at least one face.

26. The implant as claimed in claim 2, characterized in that it is present as a tube section which is designed for connection of tubular hollow organs.

27. Provision of the implant as claimed in claim 1, for an internal application in an organism, in particular in the area of wounds.

28. Provision of the implant as claimed in claim 27, the planar support being connected on both faces to an adhesive layer for apposition of vertical and horizontal tissue layers, the planar implant preferably being absorbable.
